(19) United States
(12) Patent Application Publication
(10) Pub. No.: US 2004/0101819 A1
(43) Pub. Date: May 27, 2004

(54) Self-assembled muscle-powered microdevices

(75) Inventors: Carlo D. Montemagno, Los Angeles, CA (US); Hercules P. Neves, Moorpark, CA (US)

Correspondence Address: JONES, TULLAR & COOPER, P.C., P.O. BOX 2266 EADS STATION, ARLINGTON, VA, 22202

(73) Assignee: MT Technologies, Inc., River Edge, NJ (US)

(21) Appl. No.: 10/635,933

(22) Filed: Aug. 7, 2003

Related U.S. Application Data
(60) Provisional application No. 60/401,754, filed on Aug. 8, 2002.

(51) Int. Cl. C12Q 1/00; C12M 1/34; C12N 5/06

(52) U.S. Cl. 435/4; 435/325; 435/287.1

(57) ABSTRACT

Cultured muscle tissue used as actuators in microelectromechanical systems (MEMS) for mechanical and electrical power generation can either be dissected or cultured from myoblasts and grown in situ. The MEMS is fabricated using conventional techniques (surface or bulk micromachining) and incorporating surface modification techniques and/or anchor structures to favor muscle attachment followed by post-processing to assemble dissected muscle tissue or grow the self-assembling muscle tissue at the desired sites. Initial post processing is done to incorporate PZT devices for energy conversion. Additional post-processing is then done for muscle tissue self-assembling; that includes coating the MEMS with polymers that will either repel or favor the muscle growth, and the culturing on the muscle tissue starting from myoblasts. The system is fueled by adding glucose to the medium in which it is contained.

This application claims the benefit of Provisional Application No. 60/401,754, filed Aug. 8, 2003, the disclosure of which is hereby incorporated herein by reference.

### BACKGROUND OF THE INVENTION

The present invention relates, in general, to a method and apparatus for generating electrical power from muscle tissue, and more particularly relates to the use of muscle tissue as mechanical actuators in microelectromechanical systems (MEMS) and for the generation of electrical signals.

There has been much recent activity directed toward engineering devices powered by biological structures from the molecular to the tissue level. Since individual molecular motors provide only miniscule amounts of work, the actions of millions or more must be harnessed in parallel to result in significant activity in the macroscopic world. The prospects of exploiting natural massively parallel motor assemblies, such as muscle, are very attractive since the organization, production, and manipulation of such motors from nanometer to millimeter length scales are coordinated by complex biological molecular machinery refined over millions of years of natural selection. However, extraction and integration of mature muscle tissue with mechanical devices is time-consuming and deleterious to the living components. The creation of self-assembled devices in which myocytes can selectively grow and differentiate would enable massively parallel syntheses of hybrid devices in which the biological component is perfectly healthy. To enable movement of these devices, the in situ growth and integration of muscle tissue must result in a mechanically strong attachment to the mechanical components while also permitting contraction.

As microcomponents in engineered systems, biological muscles have attractive characteristics such as large force generation, utilization of chemical fuel, and the ability to grow and self-assemble complex structures from single cells. Cardiac and skeletal muscle offer complementary capabilities: cardiac muscle can power self-triggering continuously operational devices, whereas skeletal muscle contracts only following external stimuli. Integration of either kind of muscle with microfabricated inorganic structures and electronics holds the possibility of manufacturing controllable autonomous devices powered by ubiquitous and inexpensive biomolecules such as glucose. Use of mature muscle tissue from animals in such devices is impractical, inefficient, and damaging, as the tissues must be dissected and attached individually by hand with crude interfaces between the biological tissues and inorganic materials. Incorporation of muscle with fabricated structures would be optimally achieved through directed growth in situ, since the muscle is not traumatized during device fabrication and the muscle components in multiple devices can be grown and attached in parallel.

Optical lithography has been extensively employed to pattern the growth of a variety of cell types on the micrometer scale, and the related techniques of MEMS fabrication can be used to create mechanical structures with length scales and force constants compatible with muscle tissue. However, to date there have been no reports of self-assembled muscle-powered MEMS structures, primarily due to three outstanding problems: 1) Not only must the growth of the myoctyes be spatially controlled, but the patterned myocytes must also be able to differentiate into anisotropic muscle fibers. 2) The alignment of these differentiated structures must be controlled and compatible with the surrounding mechanical structures. 3) Finally, the mature muscle tissue must be free to contract, requiring the majority of the tissue to be controllably and gently released from the substrate surface. Although several recent reports of force measurement methods describe cells integrated with micropatterned elastic substrates and cantilevers, these techniques are primarily suitable on the sub-cellular level, and do not permit free motion of the supported cells.

### SUMMARY OF THE INVENTION

The present invention meets the foregoing needs by providing a microelectromechanical structure which incorporates an anchor into which differentiated, functional muscle cells may be connected either mechanically or by growing the muscle tissue onto the anchor structure. The muscle tissue is then used as an actuator in a microelectromechanical system and this motion, in turn, may be used to provide mechanical motion or electrical signal generation. Muscle tissue for use with a MEMS structure can be dissected and mechanically connected to the MEMS device, but preferably is cultured from myoblasts and grown in situ on the device. The MEMS structure is produced by conventional surface or bulk micromachining and incorporates surface modification techniques, such as selective coating of surfaces, and/or the fabrication of anchor structures to permit muscle attachment, and the resulting device is processed to assemble dissected muscle tissue or to grow self-assembling muscle tissue at the desired sites.

The assembly of dissected tissue on a MEMS device is mainly useful for evaluation purposes, since in most cases it is not possible to preserve a functional dissected muscle tissue for very long. The preferred technique, involving the above-described on-site muscle self-assembly of muscle tissue grown from myoblasts, is a much more complex technology, but is more desirable for generation of power or mechanical motion because no manual assembly is needed for the self-assembly process and therefore, large arrays of devices operating in parallel are possible. Additionally, muscles can be grown on-site, and thus can be precisely located on the fabricated structures, with the result that finer mechanical assemblies are obtained. Further, the self-assembled muscle tissue can be preserved for a longer period of time, and finally, the self-assembly technique allows the devices to be much smaller.

One of the difficulties in using dissected tissue is the development of a suitable protocol for attaching the dissected fibers into a MEMS structure. Various mechanical attachments, such as surgical sutures, aluminum wire, and cyanoacrylate adhesive have been tried, but these all require a high level of precision. Accidental injury to the dissected tissue often results in an early loss in functionality. The use of micromanipulators for attaching the fibers is, therefore, preferred for this purpose.

In the preferred embodiment of the invention, however, functional, differentiated muscle cells are produced from a myoblast cell culture, and techniques are provided to ensure selective muscle growth without the need for human intervention to assemble such tissues in place. This form of self-assembly, which is of paramount importance, particularly for more complex MEMS structures, can be accomplished by producing spatially selective differentiation of the myoblasts by incorporating on the MEMS device materials such as polymers which either encourage or discourage muscle growth, selectively. This allows the formation of muscle tissue only on spots where they are needed, and prevents the muscles from interfering with the functionality of the MEMS structures.

In accordance with the preferred form of the invention, in situ growth, differentiation, and partial release of cells integrated on microelectromechanical systems (MEMS) substrates is effected. Implementation of these strategies with rat cardiomyocytes has resulted in the creation of the first self-assembled hybrid biotic/abiotic mechanical structures which spontaneously moved in response to the collective cooperative contraction of single mature cardiac muscle bundles. The health, morphology, and function of the cardiomyocytes integrated with these structures were indistinguishable from normal cell cultures. The lifetimes of all hybrid mechanical devices were observed to be limited not by the biological components, but by the fatigue and failure of the inorganic components. With simple initial fabricated structures, in situ studies of mechanical properties of rat cardiomyocytes, including measurements of substrate-induced cytoskeletal stress and Young's modules, have been performed. Since all types of cells and structures may be utilized, this fabrication method is highly versatile and represents a significant advance in the science and engineering of biological mechanical systems on the micro scale.

The problems identified above have been overcome by combining patterned films of the thermally responsive polymer poly(N-isopropylacrylamide) (PNIPAAm) with MEMS components. These composite substrates enable selective attachment and directed growth of rat cardiomyocytes as well as controlled release of mature muscle. Mechanical structures were fabricated using the single crystal reactive etching and metallization (SCREAM) process, to be described, from Si(111) with 1 μm of surface SiO₂. Once the Si structures were released, the entire wafer was completely covered with a solution of PNIPAAm in ethanol and dried. The final thicknesses of the PNIPAAm films ranged between 16-20 μm. Through a shadow mask, the polymer was selectively etched and coated with a Cr/Au film. Au was chosen as a growth substrate due to its excellent tensile strength, oxidation resistance, and ability to support healthy myocyte growth. The thickness of the metal film was chosen to be sufficiently thin that its bending resistance to muscle contraction was minimized, but also sufficiently thick so that it would not be destroyed during the process of polymer liquefaction described below. In sum, there are two main fabrication steps: the creation of the MEMS structures with SCREAM and the patterning of the PNIPAAm layer with Au enabling selected growth of myocytes.

In order to provide for mechanical motion and electrical signal or power generation using a muscle tissue, MEMS devices fabricated by the SCREAM process are modified by depositing on the structure a metal layer to act as an electrode, a piezoelectric film, and another metal layer to act as a second electrode. The metal used for the electrodes will vary according to the choice of the piezoelectric material. Following this tri-layer deposition, the device may be shaped, as by ion-milling, to form, for example, a capacitive strain gauge that permits quantification of the forces generated by the muscle tissue. The device can incorporate a vernier scale for visual verification of device displacement, can include springs and fingers for large displacement compatibility, and can incorporate a comb structure for capacitance measurement. The MEMS structure also includes features to facilitate the attachment of the muscle tissue, as described above.

### BRIEF DESCRIPTION OF THE DRAWINGS

The foregoing, and additional objects, features and advantages of the present invention will become apparent to those of skill in the art from a consideration of the following detailed description of preferred embodiments thereof, taken in conjunction with the accompanying drawings, in which:

FIGS. 1 and 2 are optical micrographs showing undifferentiated myoblasts (FIG. 1) and aggregated and fused myoblasts forming mytotubules (FIG. 2);

FIGS. 3 and 4 are optical micrographs showing the selective growth of myoblasts on a differentiated surface (FIG. 3) into myotubules (FIG. 4) over a period of eight (8) days;

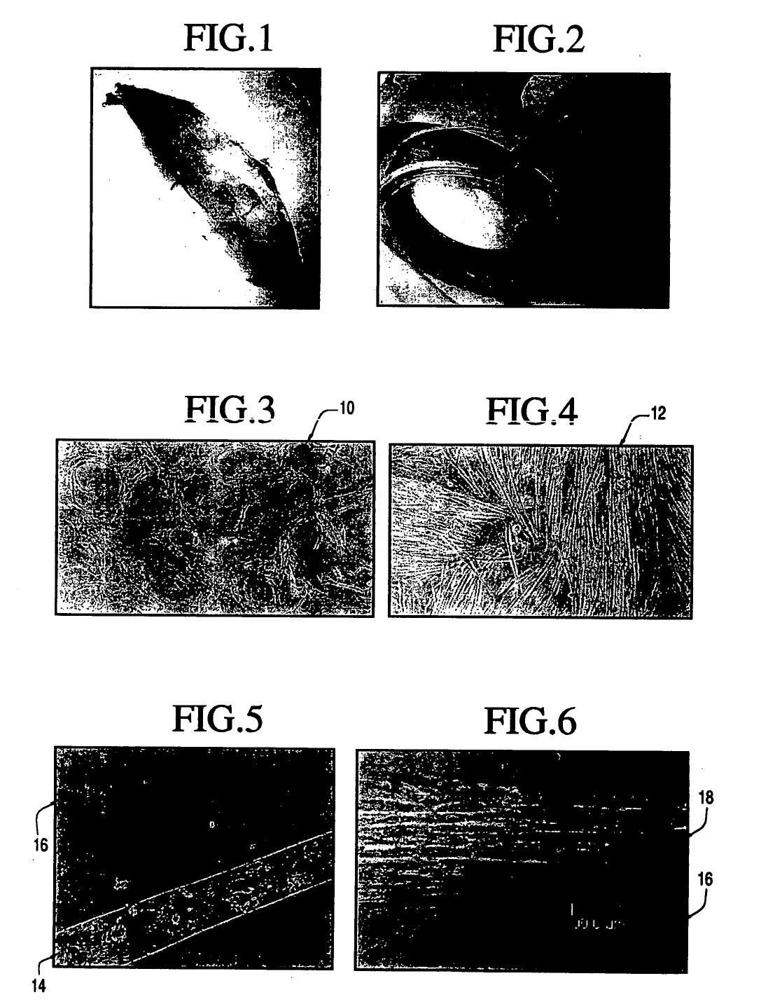

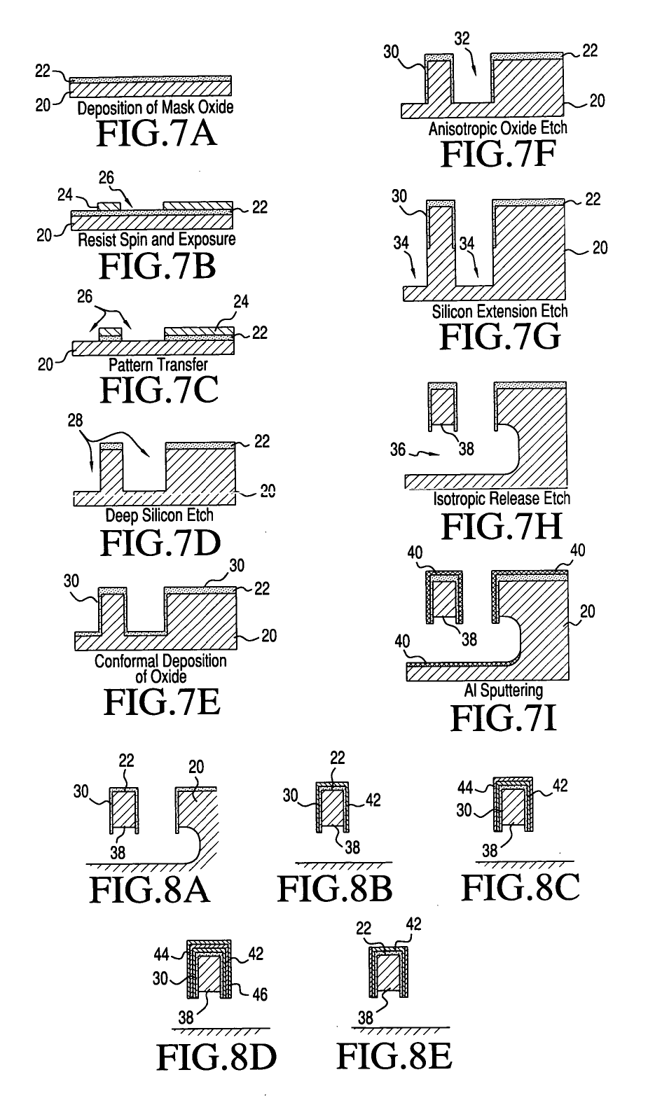

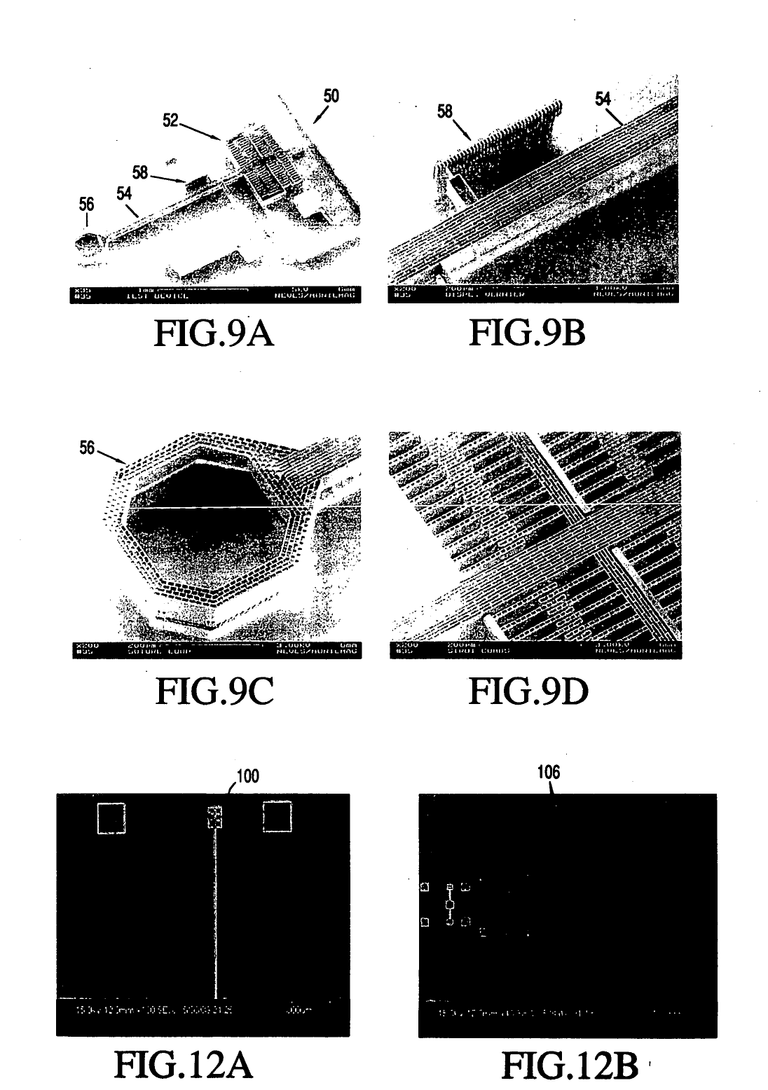

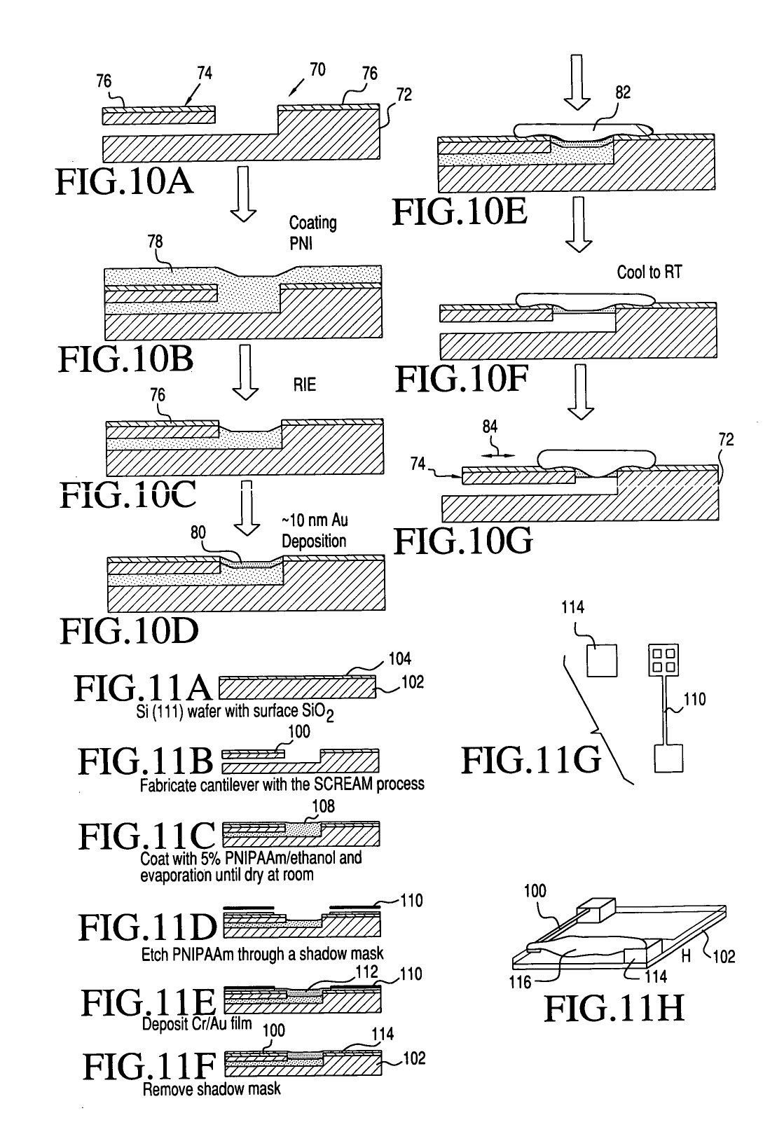

FIG. 1 is an optical micrograph showing an isolated bundle of myofibers extracted from a leg muscle;

FIG. 2 is an optical micrograph showing contractions of the myofibers of FIG. 5 under electrical stimulus;

FIGS. 7(A)-7(I) illustrate an overview of the SCREAM process used to fabricate MEMS structures;

FIGS. 8(A)-8(E) illustrate a process for fabricating a piezoelectric layer on a MEMS structure;

FIGS. 9(A)-9(D) are scanning electron microscope (SEM) micrographs of MEMS devices usable in the present invention;

FIGS. 10(A)-10(G) illustrate the steps in one embodiment of a MEMS process for accommodating muscle tissue self-attachment.

FIGS. 11(A)-11(H) illustrate a preferred embodiment of a MEMS process for accommodating muscle tissue attachment;

FIGS. 12(A) and 12(B) are photomicrographs of MEMS cantilevers;

FIGS. 13(A) and 13(B) illustrate muscle-driven cantilever motion; and

FIG. 14 is a microscopic image of a single muscle bundle.

### DESCRIPTION OF PREFERRED EMBODIMENTS

In an experiment designed to allow evaluation of the integration between muscle tissue and MEMS structures, myofibers were isolated from muscle tissue dissected from the frog Xenopus laevis. Leg muscles were excised and muscle fibers were dissected out using microsurgery apparatus. The isolated muscle bundles were further dissected to obtain individual muscle fibers. FIG. 1 shows the extracted tissue, illustrating striations of actinomyosin fibers, which indicate that the thin slice has only one layer of muscle fibers.

As shown in FIG. 2, the application of an external electrical stimulus causes the fiber to contract. To maintain their physiological activity, the fibers were stored in commercial lactated Ringer IV solution. The muscle fibers so obtained were attached to a MEMS device, such as that to be described hereinbelow, by any suitable means, such as by surgical sutures, aluminum wire, cyanoacrylate adhesive, or the like, using suitable tools such as conventional micromanipulators for the required degree of precision. Such fibers, after being connected, were actuated, as by electrical stimulation, to produce a measurable motion in the MEMS structure.

The growth of differentiated, functional muscle cells from myoblasts is illustrated in FIGS. 3-6. In an example of the process of the invention, a myoblast cell culture was selected from the C2C12 cell line. Such myoblasts, shown at 10 in the optical micrograph of FIG. 3, were cultured in Dulbecco's Modified Eagle's Medium (DMEM) containing 10% fetal calf serum (FCS) depleted of the thyroid hormones at 37° C. No differentiation or fusion into myotubules was observed under such conditions, as shown in the figure.

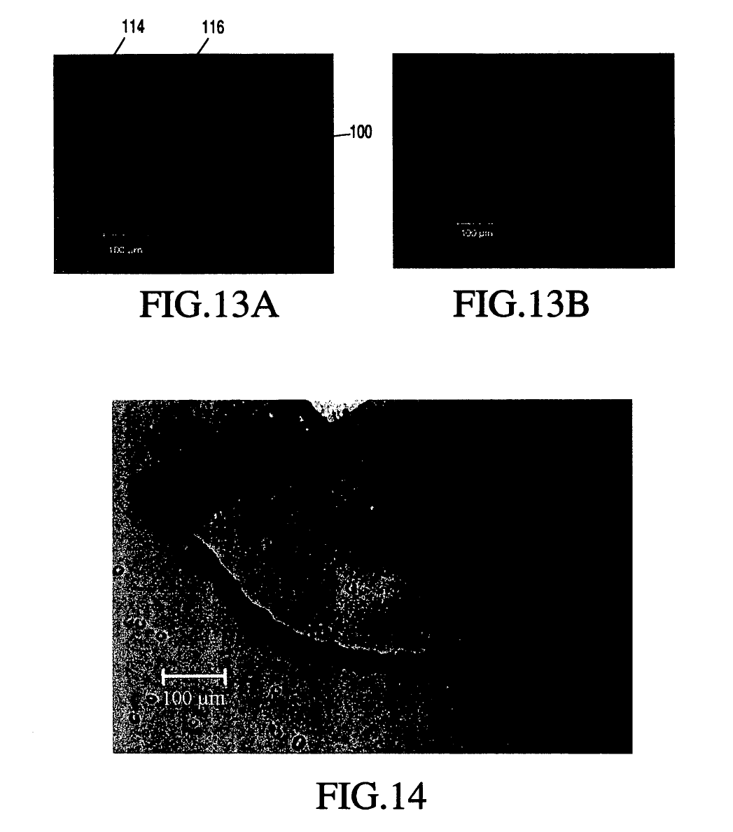

In the experiment, after a period of four days, the tissue culture medium was switched to 2% horse serum in DMEM, and the cells became aggregated or fused to form myotubules, as illustrated at 12 in FIG. 4.

For successful integration between MEMS structures and muscle cells or other tissues grown in vitro, it is crucial to ensure selective muscle growth without the need for human intervention to secure the tissues in place. This selective growth is a form of self-assembly of the MEMS and muscle combination, and is of paramount importance for complex MEMS devices. Due to the nature of the tissues used in the present method and the resulting devices, the need for selective growth must be taken into account from the beginning of the process, so that a spatially selective differentiation of myoblasts into myotubules can be obtained.

In order to obtain such a selective differentiation, so as to enable connection of the tissue to selected regions of a MEMS device, a number of materials have been identified on which myotubule growth was either highly efficient, or was negligible. A number of polymers which provided these characteristics include Polydimethylsiloxane and Sylgard 184, which showed no cell growth after three days, Polysulfone, Polycaprolactone, Polyhydroxybutyrate, Laminin, Gelatin, and Collagen, which showed very good cell growth after three days, and Matrigel, which showed modest cell growth after three days.

Among the tested compounds, polydimethylsiloxane (PDMS) showed good results. For example, samples that contained PDMS adjacent to growth-favorable polymers (such as polysulfone) showed an almost total absence of cells on the PDMS, while cells remained attached and even grew into differentiated myotubules on the adjacent regions, as shown in FIG. 5. In this figure, a polycaprolactone strip 14 was patterned onto a PDMS-coated substrate 16, and the substrate was submitted to the culture medium. As shown, there was a total absence of cells on the exposed PDMS surface 16, while normal growth and differentiation is shown on the polycaprolactone-coated line 14. Over a period of eight (8) days, the cells on strip 14 grew into myotubules, shown at 18 in FIG. 6.

PNIPAAm, a thermally responsive polymer, has been previously considered as an intelligent substrate to pattern cells. A solid at temperatures greater than 32° C., PNIPAAm undergoes a solid-liquid phase transition as it is cooled to lower temperatures and can dissolve in a surrounding liquid medium. In another experiment, cardiomyocytes grew well on Au films, but rather poorly on PNIPAAm. By overcoating MEMS structures with PNIPAAM, selectively etching the PNIPAAm, and patterning Au on the PNIPAAm, cell cultures grown on the entire device result in monolithic muscle structures only on the Au and are directly supported by both Si and Au/Cr/PNIPAAm. Polymer etching prior to the metal film deposition ensures that the ends of the Au film will be directly on the cantilever and on the solid support. However, after the self-assembled devices are cooled, the polymer liquefies and dissolves, releasing selected regions of the muscles and allowing them to freely contract. Furthermore, the dissolution of the polymer also releases any cells which have adhered to the polymer, although unhealthily, in unintended locations. The temperature response and the myocyte growth inhibition of PNIPAAm make it an ideal negative material to pattern the myocytes. The other roles played by PNIPAAM are to support the Cr/Au film, to protect the cantilever during the period of myocyte culture, and to prevent the released cantilever from sticking to the underlying surface from surface tension during cell culture.

The ability to selectively form muscle tissue on particular materials allows such tissue to be connected to selectively coated MEMS structures, without interfering with the functionality of such structures.

Suitable MEMS devices may be fabricated using known fabrication techniques. A bulk micromachining process is preferred, however, for compared to surface micromachining, the bulk process can produce greater distances between movable MEMS structures and the substrate on which they are mounted. These greater distances are advantageous for the self-assembly of muscle tissue on the MEMS device. The bulk process also leads to much higher aspect ratios (the ratio of structure height to width), making such structures more rugged and able to withstand the manipulations required if manual integration of muscle fibers and MEMS structures is to be used. A preferred process is the Single Crystal Reactive Etching and Metallization (SCREAM) process, developed at Cornell University, and described, for example, in U.S. Pat. No. 5,846,849, the disclosure of which is hereby incorporated herein by reference. It will be understood, however, that other types of bulk micromachining can be used.

The single-mask SCREAM process is illustrated in FIGS. 7A-7I, wherein a single crystal silicon substrate 20 is initially coated with a layer of Plasma-Enhanced Chemical Vapor Deposition (PECVD) silicon dioxide 22, which is used as a hard mask for subsequent silicon patterning. A photoresist layer 24 is then spun onto the top surface of mask layer 22 and photolithography is performed to define the required patterns 26, as illustrated in FIG. 7B. The patterns 26 are then transferred into the mask layer 22, as illustrated in FIG. 7C, and then into the silicon substrate 20, as illustrated at 28 in FIG. 7D, using Deep Reactive Ion Etching (DRIE). This Deep Reactive Ion Etching consists of a more aggressive type of RIE, in which the plasma is inductively coupled, thus eliminating the Debye shielding. To better control the profile of such etching, the process is performed with alternating etching and polymer deposition steps. Once the desired depth is achieved, which in the final device will dictate the height of the moving structures, the surface is once again coating with a conformal layer 30 of PECVD silicon dioxide to protect the side walls.

Thereafter, a short RIE step is done to remove the silicon dioxide layer 30 from the floor of the etched pattern, as indicated at 32 in FIG. 7F, and another DRIE step is done, as illustrated at 34 in FIG. 7G, to extend the depth of the structure. This defines the ultimate distance between the moveable structures and the substrate.

A high pressure DRIE etch is done to isotropically etch the substrate as illustrated in FIG. 7H to undercut the narrow structures at 36 and to release them from the substrate, as indicated by released beam structure 38, while the wider structures will not be undercut and will remain attached to the substrate. Finally, interconnects and power-generating films are deposited on the structure, as illustrated in FIG. 7I by the layer 40 which overlies the stationary substrate 20 and the moveable beam structure 38.

The process for fabricating the layer 40 is illustrated in greater detail in FIGS. 8A-8E, to which reference is now made. Once the suspended MEMS structures are fabricated, as illustrated in FIG. 8A by movable beam structure 38, carrying silicon dioxide layers 22 and 30, a metal deposition is performed to produce a first electrode layer 42(FIG. 8B). Thereafter, a piezoelectric film 44 is deposited, illustrated in FIG. 8C, and this is followed by the deposition of a second metallic layer 46, which forms a second metallic electrode, illustrated in FIG. 8D. Ion milling is then performed, which causes the second electrode layer 46 and the piezo material layer 44 to be removed from the tops of the structures, as illustrated in FIG. 8E, leaving the triple layer intact on the sidewalls. The structures may then be connected together, as needed, by way of the first electrode on the top surface. The metals used for these layers may vary according to the choice of piezoelectric material. For example, for lead-zirconium-titanate (PZT) piezoelectric layer, a platinum metal layer is usually employed. As far as the choice of piezoelectric material, ZnO, and polyvinyllidineflouride (PVDF) have been used in addition to PZT.

A MEMS motion sensor 50 is illustrated in FIGS. 9A-9D and includes a capacitive strain gauge 52 in the form of a comb structure for muscle tissue force measurement. The sensor includes a moveable released MEMS beam 54 which includes at its free end a loop 56 for receiving sutures, wires, or the like for securing a muscle fiber to the sensor device 50. The suture loop 56 is shown in an enlarged view in FIG. 9C. A visual vernier scale 58, illustrated in an enlarged view in FIG. 9B, is located adjacent the beam 54 to provide fast visual verification of beam displacement, while the capacitive comb structure 52 illustrated in an enlarged view in FIG. 9D, incorporates movable and stationary interdigitated fingers for measuring the motion of the beam. Such motion may vary the capacitance between the adjacent fingers, or, if the fingers are oppositely charged, may produce a corresponding electrical current representing the motion of the muscle fiber.

A modification of the MEMS fabrication process to permit self-assembly of muscle fibers in accordance with one embodiment of the invention is illustrated in FIGS. 10A-10G, to which reference is now made. As illustrated, bulk micromachining is used in the manner described above with respect to FIGS. 7A-7I, resulting in the structure 70 of FIG. 10A, which includes a stationary substrate 72 and illustrates a single moveable beam 74 which maybe, for example, a cantilever area. In the present embodiment, the released beam may be shallower; i.e., may have a lower aspect ratio, than the device illustrated in FIGS. 9A-9D.

In the process illustrated in FIGS. 9A-9D, the finished, released MEMS structure is top coated with a layer of gold 76, which may be thermally or e-beam evaporated onto the top surfaces. Thereafter, as illustrated in FIG. 10B, a layer of a suitable polymer such as PNIPAAm is spun onto the structure to provide a layer 78. Although PNIPAAm is preferred, similar polymers can be utilized, if desired. This polymer, however, has the advantage that it offers mechanical support for cell growth and can be safely removed from the structure without killing the cells. PNIPAAm remains a solid unless it is exposed to water at temperatures lower than 30° C., and can therefore withstand post-MEMS processing and cell culture, which occurs at temperatures around 37° C., and can be dissolved by simply lowering the system temperature. Because of its 30° C. threshold, cells are not affected in the process of dissolving it.

As illustrated in FIG. 10C, reactive ion etching is performed to expose the top surfaces of the structure, and therefore to expose the top gold film 76. Thereafter, a second gold deposition is performed, as illustrated at 80 in FIG. 10D, and muscle tissue 82 is grown on the site, as illustrated in FIG. 10E. Thereafter, the PNIPAAm is sacrificially removed by lowering the system temperature, as illustrated in FIG. 10F, without affecting the muscle structure, and as illustrated in FIG. 10G, the muscle tissue 82 then spans the distance between the stationary structure 72 and the movable structure 74 so that stimulation of the muscle causes a relative motion between structural components 72 and 74, as indicated by arrow 84 in FIG. 10G.

In another embodiment of the invention, illustrated in FIGS. 11A-11H and in the photomicrographs of FIGS. 12A and 12B, cantilevers, such as cantilever 100, were fabricated from a 4-inch Si (III) wafer 102 having a 1 μm layer 104 of surface thermal SiO₂, using the SCREAM process described above. As illustrated at 106 in FIG. 12B, in the experiment multiple cantilevers of differing lengths were fabricated, and released, although the following description refers only to one of them. Thereafter, the entire wafer 102, and its released cantilever 100, was completely covered with a 5% solution (weight/volume) of PNIPAAm 108 (produced by Polyscience, Inc.) in ethanol, followed by exposure to the air until totally dry (FIG. 11C). The final thickness of the PNIPAAm film was measured by a TENCOR Alpha-stop 200, and ranged between 16-20 μm. The wafer was then baked at 80° C. for 10 minutes, followed by selective thinning of this polymer using oxygen RIE etching through a hard shadow mask 110 (FIG. 11D). After that, a 60 μm wide, 25 nm thick adhesive layer of Cr and 300 nm of Au were deposited as a layer 112, using e-beam evaporation through the same shadow mask (FIG. 11E) and the mask was removed (FIG. 11F).

As shown in FIG. 12B, cantilevers 106 with lengths ranging from 100 to 500 μm were fabricated to measure the stress and strain of the contraction of single muscle bundles. The spring constants of the cantilevers were calibrated directly using a bent glass fiber, the spring constant of which was measured directly. Wafers containing these structures were incubated at 37° C. in cell culture medium containing neonatal rat ventricular cardiomyocytes. At 37° C., the PNIPAAM is a solid gel, supporting the Cr/Au film and providing a stable matrix for the mechanical components. Following 2-3 days of culturing, the myocytes grew on the Au film, showing no obvious difference from those grown on normal petri dishes, while negligibly present on the PNIPAAm surface. The Au 112 film defining the extent of the muscle bundles spanned from the end of the cantilever 100 to a solid support 114 (FIGS. 11F-G). The middle region of the Au film 112 was supported by the polymer and therefore was suspended after the polymer dissolution. Within 10 minutes following removal to room temperature, the polymer liquefied and dissolved in the surrounding medium, leaving the cardiac muscle bundles illustrated at 116 in FIG. 11H free to spontaneously contract, which was observed through microscopic observation of rhythmic bending of the cantilever beams illustrated in FIGS. 13A and 13B.

Observations showed that, upon cooling and release, the cantilever beam 100 exhibits two distinct states, sequentially: 1) an static resting state where a static force deflects the cantilever, seen at all times when the muscle is not contracting; and 2) A power stroke resulting from muscle contraction, where the cantilever deflection increases to a maximum and quickly returns to the static state, ready to repeat. The contraction cycles were monitored for the entire lifetime of the device (˜1-2 hours), which was always limited by failure of the MEMS cantilever at its base. Initial data showed that the maximum cantilever deflection produced by individual muscle bundle strokes was very consistent over time, varying less than 6% over the course of observation. The maximum deflection amplitudes varied with cantilever length, indicating that they were force-limited. Two characteristic times of the resultant motion, the time of contraction and the time between contractions, were also measured, and both of these times increased with increasing stroke number, indicating fatigue of the biological component.

The static deflection state indicates a force balance between the bent cantilever and the released muscle bundle. The static force from the released bundle was due to cytoskeletal stress induced by cellular surface adhesion during growth on the gold surface. This was verified by substituting epithelial cells and rat fibroblasts for myocytes and proceeding with normal culture conditions. During cell culture on the surfaces of Petri dishes and Au films, similar spreading morphologies were observed for both the epithelial cells and fibroblasts. When these cells were grown on the Au films and released, no rhythmic contraction was observed, but only a static curvature similar to that seen with the myocytes (FIG. 14).

To quantitatively measure the tissue stress producing this force requires measurement of the thickness of the muscle bundles. Gold films which were unattached to any MEMS structures resulted in completely free bundles, the thickness of which could be easily measured. The average measured thickness of muscle bundles was 29.1±2.7 μm. It was assumed that the muscle bundles have the same cytoskeletal strain when grown under the same conditions and that the cytoskeleton provides a restoring force linear with displacement. Further, observations also showed that the strain of muscle bundles grown under the same conditions is constant, indicated by the same curvature of muscle Cr/Au film after the release. The polymer coating and etching process described above results in a trough-shaped profile of the polymer between the cantilever and the solid support. Therefore the Cr/Au film will not buckle as the muscle bundle contracts, but instead bends with negligible resistance.

Analysis of the static force balance results in determination of a cytoskeletal Young's modulus of 40 kPa and a surface adhesion stress of ˜2-2.5 kPa. As cytoskeletal forces and the mechanical interaction between cells and their substrates are known to play a critical role in many cellular events such as cell locomotion, embryonic development^{16,17}, tissue growth, and wound healing, the method described here for measurement of these forces will be particularly useful, since the cytoskeletal stress and cell-substrate adhesion can be probed and characterized for almost all cells and surfaces in a highly non-invasive manner.

A similar analysis can result in the maximum force exerted by the muscle bundles during a contraction stroke, since the peak contraction force is balanced by the cytoskeletal stress and the cantilever restoring force. In the experiments discussed here, the average peak contraction force was 30.0 μN and 48.6 μN and the contraction stress was 11.7 and 17.9 kPa with the 400 and 200 μm-long cantilevers, respectively. The fully contracted strains were −13.8% and −5.7% relative to the length after release and prior to the contraction. These two groups of values reflect the difference of the myocytes under the different loading forces and stresses, which were 19.36 μN, 38.36 μN, 7.56 kPa, 14.2 kPa, respectively, and have been shown to alter the magnitude of the peak contraction force. Both the contraction time and the time between contractions also significantly varied under the different loading forces. Therefore, by varying the dimensions of the fabricated cantilevers, the muscle pre-load may be systematically varied and the resultant stroke force measured completely non-invasively. Further, the dynamic properties of muscle contraction can also be monitored using these devices.

The shapes of the muscle bundles are dependent only on the pattern of the gold film (or on the pattern of any other substance conducive to cell adhesion and growth), and therefore can be tailored arbitrarily to the shapes, sizes, and geometries desired. Since the forces produced by the muscle bundles are proportional to their lateral dimensions, they may be specified simply by changing the width of the underlying gold film. Since the basic principles of tissue patterning and release discussed above are also applicable to skeletal muscle, integration of electronic components into the fabricated MEMS structures leads to the possibility of triggered muscle contraction and coordinated movement of multiple separate muscle components of a single device.

In short, a self-assembled muscle-based micro-transducer system has been developed. This system is capable of patterning and controlling differentiation of myocytes and controlling the initiation of device activity. Preliminary investigations of this system have demonstrated its applicability for study of in situ mechanical properties of both skeletal and cardiac myocytes, as well as measurements of cytoskeletal stress and strain and surface adhesion forces. Improved knowledge of the static and dynamic characteristics of cardiomyocytes would contribute to better understanding of cardio tissue physiology and further engineering of functional cardiac tissue constructs. Further, since MEMS structures are able to be completely released from the surface, fully autonomous mobile structures can be constructed with these techniques which can be powered by any glucose-containing medium such as blood.

The cantilever dimensions were measured using a scanning electron microscope (Hitachi S4700). The spring constants of the cantilevers were calculated based on these dimensions using k=Ehw³/41^{3 18}. These calculations were verified by direct measurement of a small number of cantilevers as follows: the spring constant of a drawn glass fiber was determined by hanging a number of weights from its end and measuring the deflection microscopically. The fiber was then used to deflect the MEMS cantilevers and the deflections of both the fiber and cantilever were measured, resulting in the spring constant of the cantilever.

Cell culture and differentiation of neonatal ventricular myocytes was carried out using 1-3-day-old Sprague-Dawley rats (NRVMs). The cell cultural medium (NRVMs) and conditions were conventional, and prior to the isolated myocytes being plated, the fabricated devices already glued to ordinary culture dishes were warmed to 37° C. The plated myocytes density is 4.6-6.1 million/cm² and this culture would be kept at a 37° C. incubator supplied with 5% CO₂ for 2-3 days.

Strain and thickness measurement of single muscle bundle was carried out using a process similar to the muscle-MEMS devices described above. Patterned Au films were deposited over a layer of PNIPAAm, with the exception that the Au films did not touch any Si surfaces. After PNIPAAm complete dryness, 100-nm-thick gold films with different widths were deposited via a shadow mask. The same culture condition as above described was applied to plate the myocytes on gold film. After the differentiation and maturation of the muscle bundles, the polymer was cooled to dissolve into the culture medium. The unattached muscle and gold drifted in the medium, which was gently agitated until the bundle was oriented with the gold film perpendicular to the planar surface (FIG. 14). The length and thickness were then measured microscopically.

The imaging system was composed of a microscope (Nikon E800) with a CCD camera (Hamamatsu C240) and a videocassette recorder (Sony DVCAM DSR-30) and mounted on an air-suspension table. After the culture finished, the petri dish containing the devices was placed at room temperature under a microscope for imaging and analysis. The digitized images were transferred to a PC computer and were subsequently contrast enhanced, and analyzed on a pixel basis to obtain the bending distance and the thickness of single bundle myocytes.

Thus, there has been described unique methods for attaching muscle tissue to a movable microstructure to enable a muscle to produce motion in such devices. The motion can be sensed, to detect motion, or can be used to generate electrical signals, or electrical power. The technique allows fabrication of large numbers of muscle-driven microelectromechanical structures to allow production of significant levels of power, yet permits fabrication of minute devices sufficiently sensitive to detect small amounts of motion. Although the invention has been described in terms of preferred embodiments, it will be apparent that numerous modifications and variations may be made without departing from the true spirit and scope thereof, as set forth in the following claims.

What is claimed is:

1. A method of growing muscle tissue on preexisting mechanical structures in such a way that the tissue is firmly anchored to the structure yet free to contract, and a method of generating electrical power from muscle tissue, comprising:
fabricating a MEMS device incorporating an anchor which favors muscle attachment;
recurring muscle tissue to said anchor; and
detecting muscle motion at said MEMS device by generating a corresponding electrical signal.

2. The method of claim 1, wherein fabricating said anchor includes shaping said MEMS device to produce a structural feature for receding a fastener to connect said tissue to said anchor.

3. The method of claim 2, wherein fabricating said anchor includes shaping said MEMS device to provide a loop for receiving said fastener.

4. The method of claim 1, wherein securing muscle tissue to said anchor includes assembling said muscle tissue to said anchor.

5. The method of claim 1, wherein recurring muscle tissue to said anchor includes self-assembling said muscle tissue on said anchor.

6. The method of claim 5, wherein self-assembling said muscle tissue includes growing muscle tissue on said anchor.

7. The method of claim 6, wherein self-assembling said muscle tissue includes differentially treating said MEMS device to limit the growth of muscle tissue to selected regions of said MEMS device.

8. Apparatus for producing electrical signals from muscle tissue, comprising:
a microelectromechanical structure comprising a substrate and a released structure relatively movable with respect to said substrate;
an anchor on said released structure for receiving muscle tissue; and
a motion sensor responsive to motion of said anchor produced by muscle contraction for producing a corresponding electrical signal.

9. The apparatus of claim 8, wherein said anchor includes means for assembling said tissue to said released structure.

10. The apparatus of claim 9, wherein said means from assembling comprises an aperture for receiving a tissue fastener.

11. The apparatus of claim 8, wherein said anchor includes means for self-assembling said tissue to said released structure.

12. The apparatus of claim 11, wherein said means for self-assembling comprises differentially treated portions of said microelectromechanical device to promote the growth of said tissue on selected regions of said devices.

13. The apparatus of claim 12, wherein said differentially treated regions include a growth-supporting material on said selected regions.

14. The apparatus of claim 13, wherein said growth-supporting material is a polymer.

15. The apparatus of claim 13, wherein said differentially treated regions are located on both said substrate and said released structure to permit said tissue to span between said selectively movable substrate and released structure to enable contraction of said tissue to produce relative motion therebetween.
